(19) United States
(12) Patent Application Publication
(10) Pub. No.: US 2016/0242867 A1
(43) Pub. Date: Aug. 25, 2016

(54) Wound Management Device

(71) Applicant: SALTS HEALTHCARE LIMITED, Aston, Horsham, West Sussex (GB)

(72) Inventors: Eric Davison, Horsham, West Sussex (GB); Aikatarini Mouliadou, Horsham, West Sussex (GB); Neil Wiltshire, Horsham, West Sussex (GB)

(21) Appl. No.: 15/026,812

(22) PCT Filed: Sep. 26, 2014
(86) PCT No.: PCT/GB2014/052919

§ 371 Date: Apr. 1, 2016

(30) Foreign Application Priority Data
Oct. 2, 2013 (GB) 1317459.4

(51) Int. Cl. A61B 90/40; A61M 27/00; A61F 15/00

(52) U.S. Cl. CPC A61B 90/40; A61F 15/008; A61M 27/00; A61B 2090/401

(57) ABSTRACT

A wound management device including:
at least one securing element for securing to skin around a wound site;
a chamber defining member connected to the at least one securing element for defining a chamber wherein the wound is accessible;
wherein an opening is provided in the chamber so that access can be gained to the chamber, said opening being moveable between closed and open conditions, and there being at least one member for holding the opening in its open condition.

This invention relates to a wound management device.

Treatment of wounds can involve the use of dressings which fulfil the basic functions of covering a wound to keep it clean and protect it during healing. It may also be required to facilitate the delivery of one or more substances to the wound site and/or the removal of one or more substances from the site. For example, fluids such as liquids or gases may be supplied to or carried away from the wound site. Liquid fluids such as irrigation or cleansing agents, e.g. saline, or gases such as oxygen for aeration of the wound site may be supplied thereto. After use such fluids, in addition to wound exudates, may be conducted away from the wound site. Wound management devices, which sometimes are referred to as wound pouches, provide for such handling of fluids.

Wound management devices are also used for managing exudates from fistulas. In referring to the present invention as a wound management device, it is to be understood that it will find utility for such other purposes, where similar or analogous requirements may arise.

When a wound management device is to be used for the supply to and/or removal from a wound site of one or more fluids, it must provide for the connection of one or more fluid conduits, such as flexible tubing, to the device in such a way as to communicate with the wound site to which the device is applied. To this end it has been usual to provide a wound management device with an entry portion provided with or able to receive, at least one element affording a through-passage by which fluid can enter or leave a space defined by the device, for access to the wound.

According to the present invention we provide a wound management device including:
at least one securing element for securing to skin around a wound site;
a chamber defining member connected to the at least one securing element for defining a chamber wherein the wound is accessible;
wherein an opening is provided in the chamber so that access can be gained to the chamber, said opening being moveable between closed and open conditions, and there being at least one member for holding the opening in its open condition.

Further features of the invention as set out in dependent claims 2 to 20 appended hereto.

Embodiments of the invention will now be described by way example, with reference to the accompanying drawings, of which:

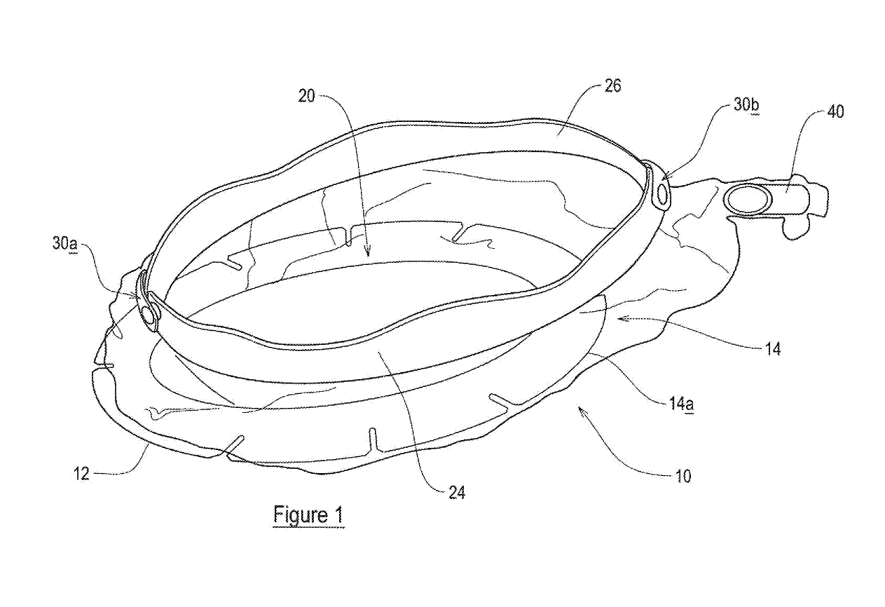

FIG. 1 is a perspective view of a wound management device in accordance with the present invention in an open condition;

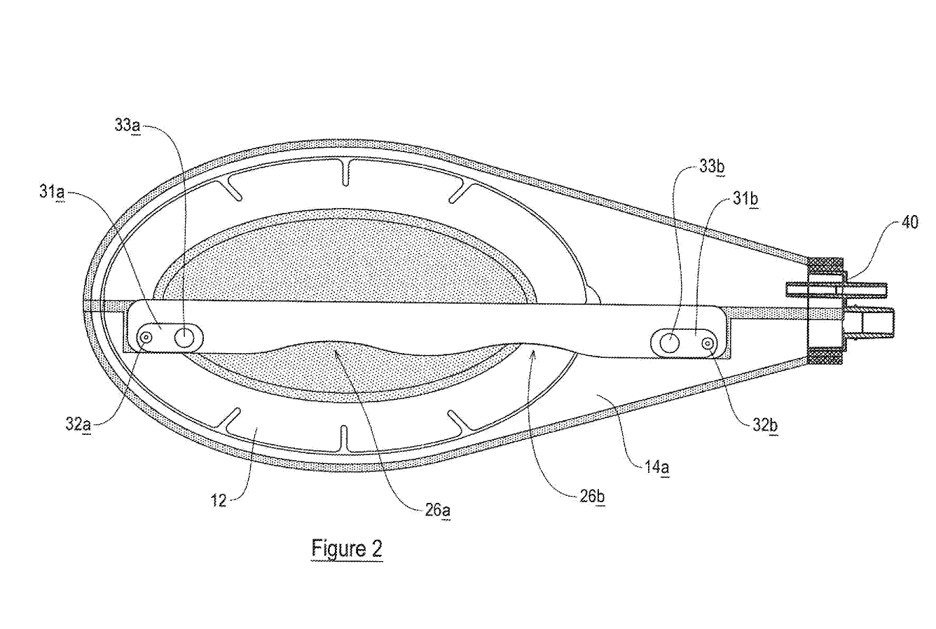

FIG. 2 is a plan view of the device of FIG. 1 in a closed condition;

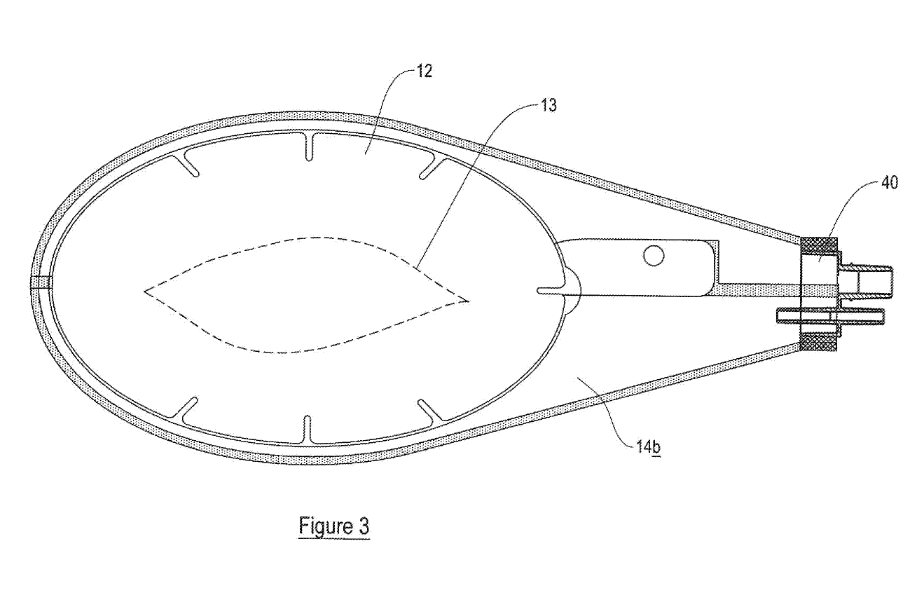

FIG. 3 is a an underside view corresponding to FIG. 2;

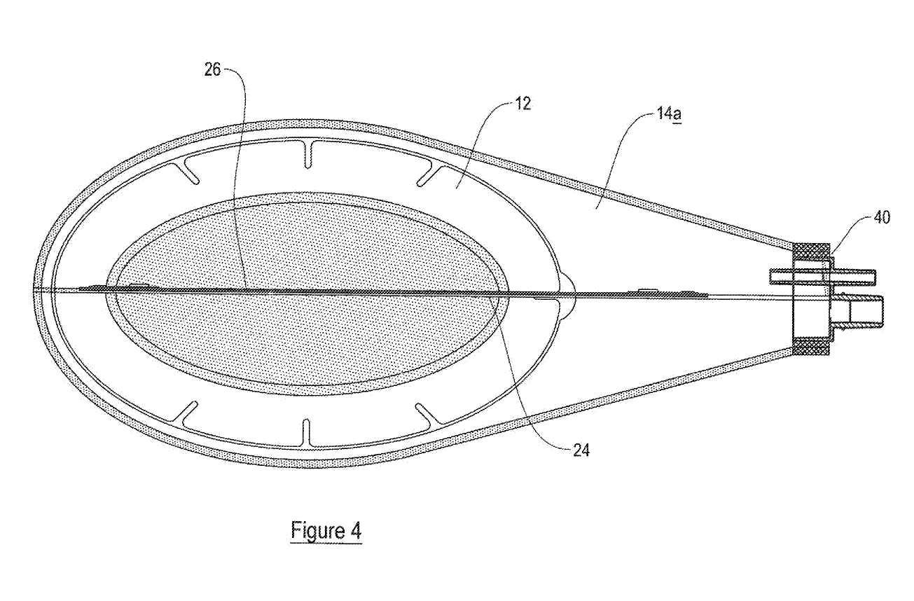

FIG. 4 is a further plan view of the device;

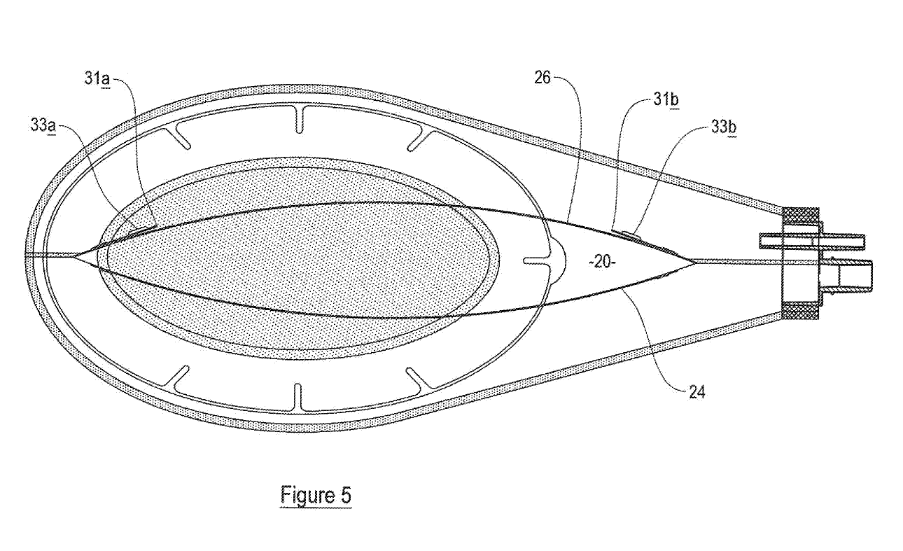

FIG. 5 is a yet further plan view of the device in a partially open condition;

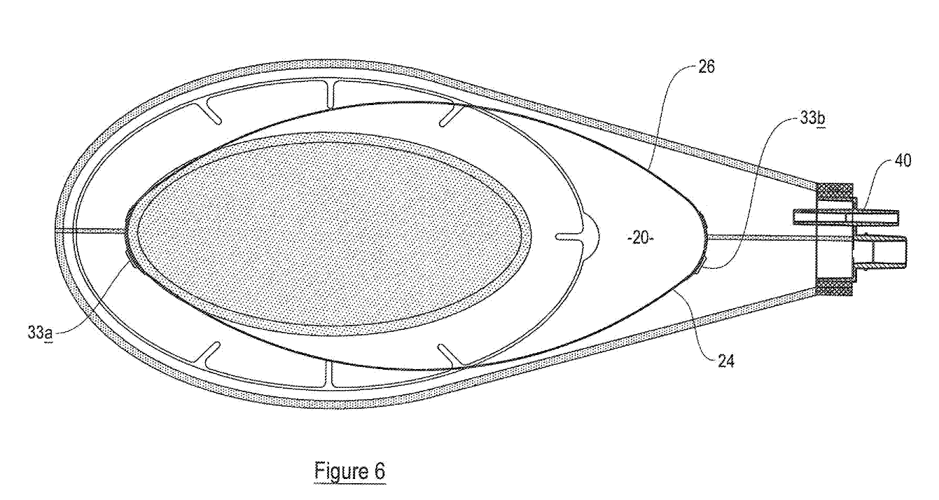

FIG. 6 is a yet further plan view of the device in an open condition;

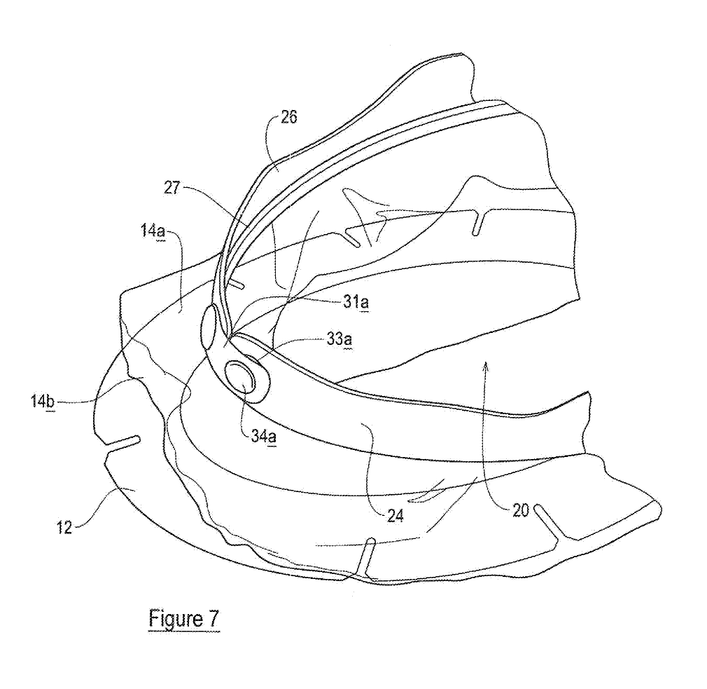

FIG. 7 is a close up side perspective view of component parts of the device of FIG. 1;

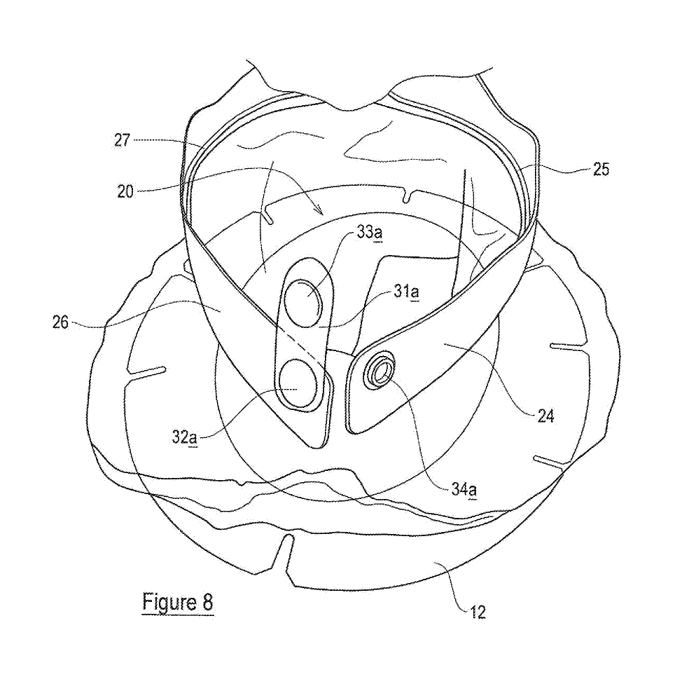

FIG. 8 is a close up end perspective view of component parts of the device of FIG. 1;

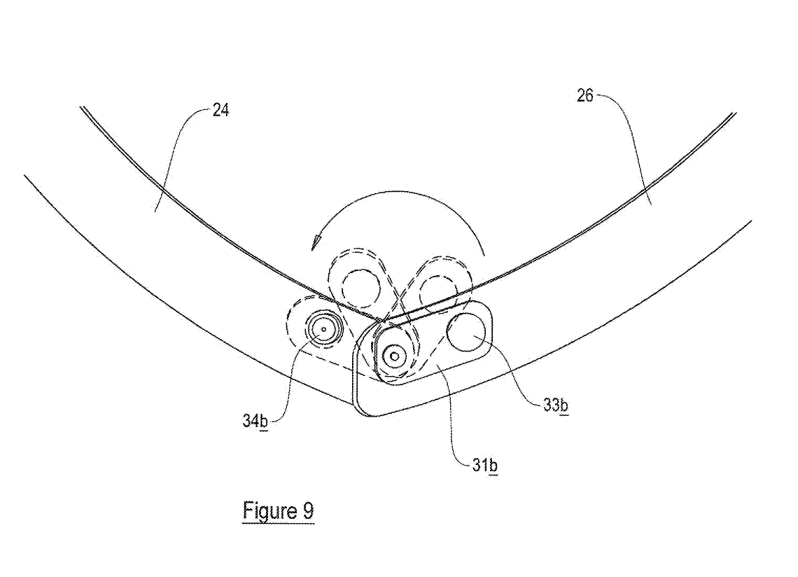

FIG. 9 is a further close up end perspective view of component parts of the device of FIG. 1;

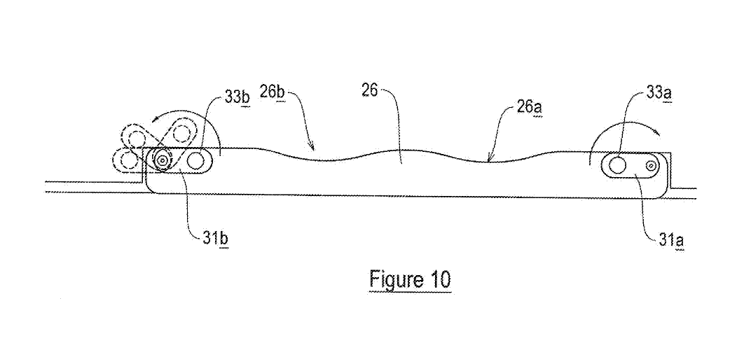

FIG. 10 is a side view of a chamber opening support member of the device;

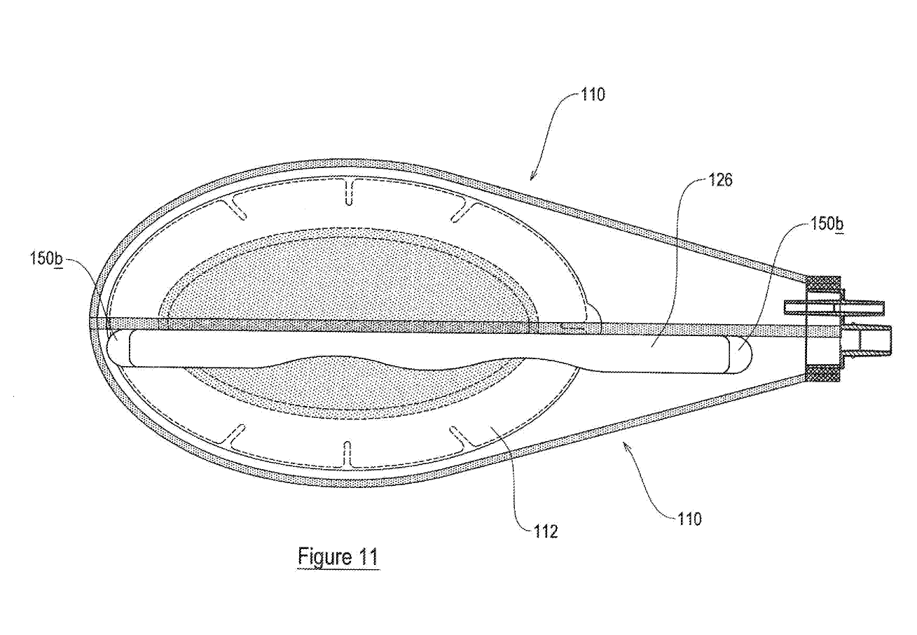

FIG. 11 is plan view of a second embodiment of a device in accordance with the present invention;

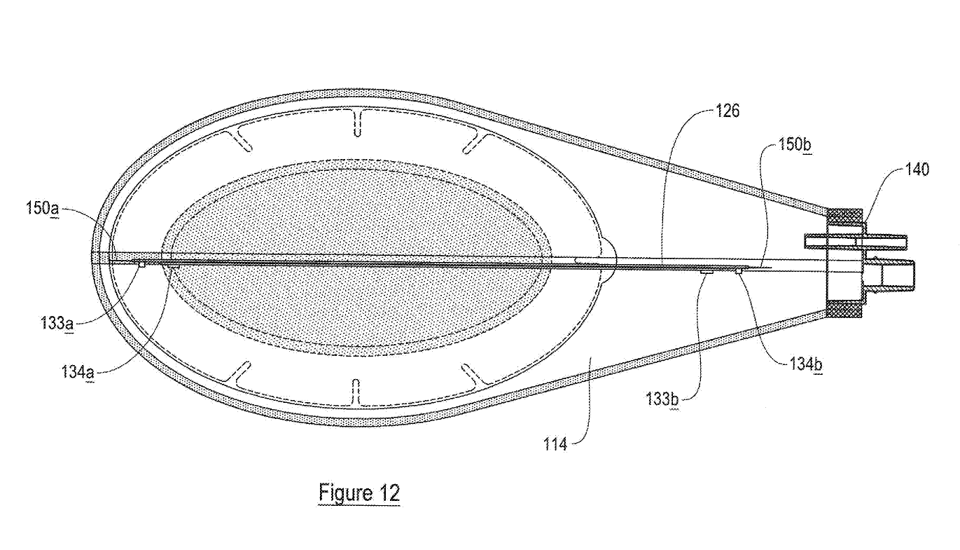

FIG. 12 is a further plan view of the device of FIG. 11;

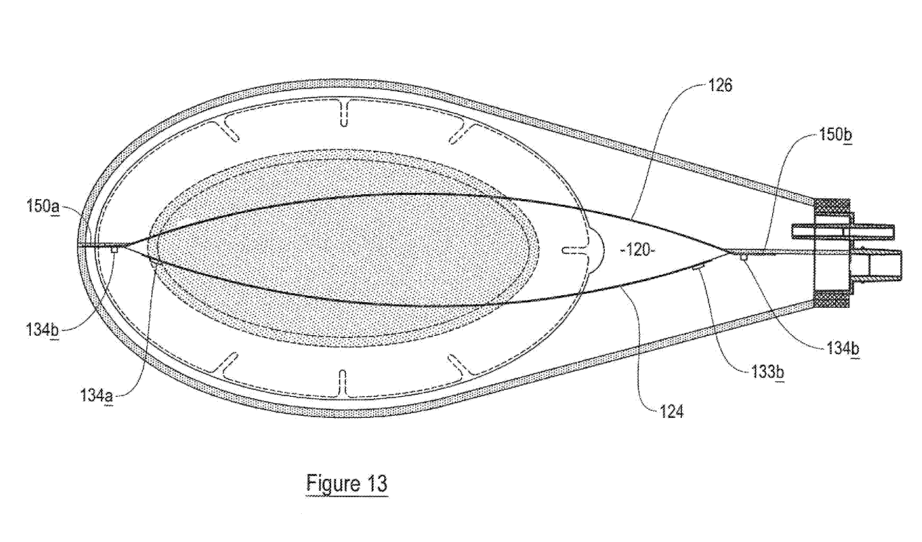

FIG. 13 is a yet further plan view of the device of FIG. 11 in a partially open condition;

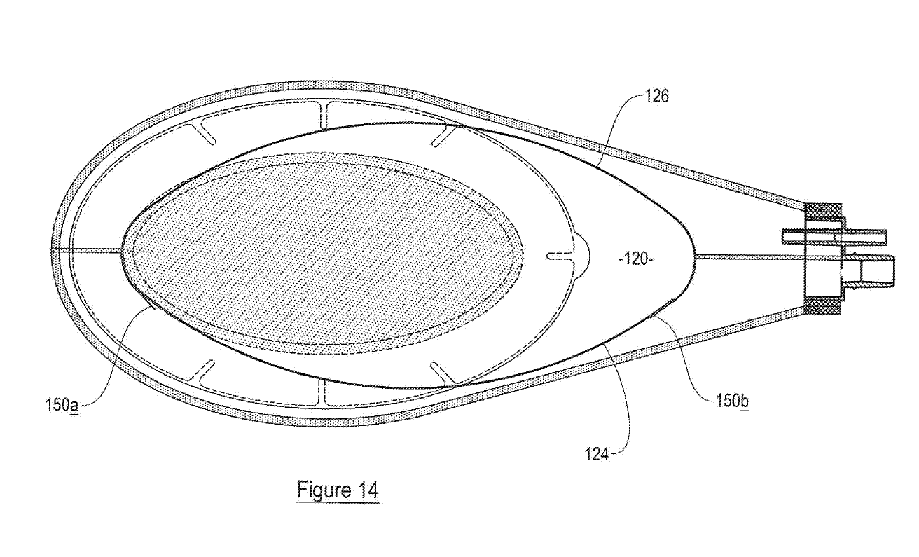

FIG. 14 is a yet further plan view of the device of FIG. 11 in an open condition;

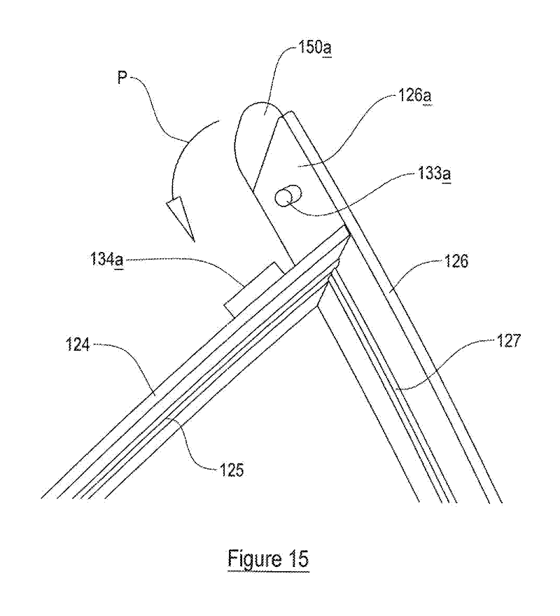

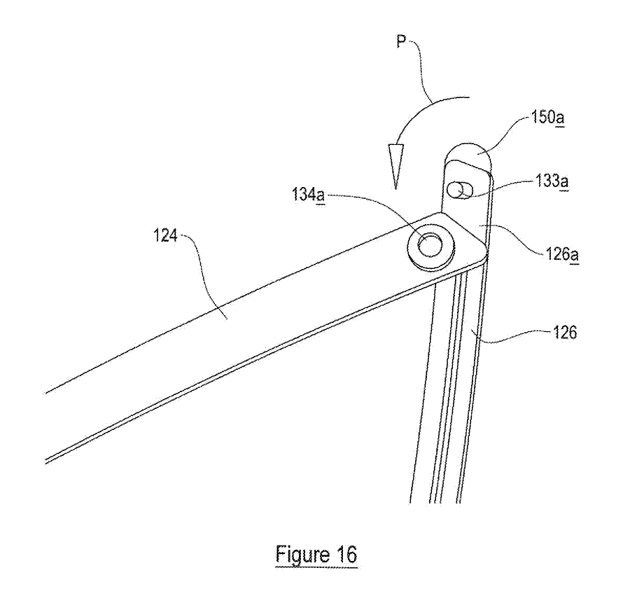

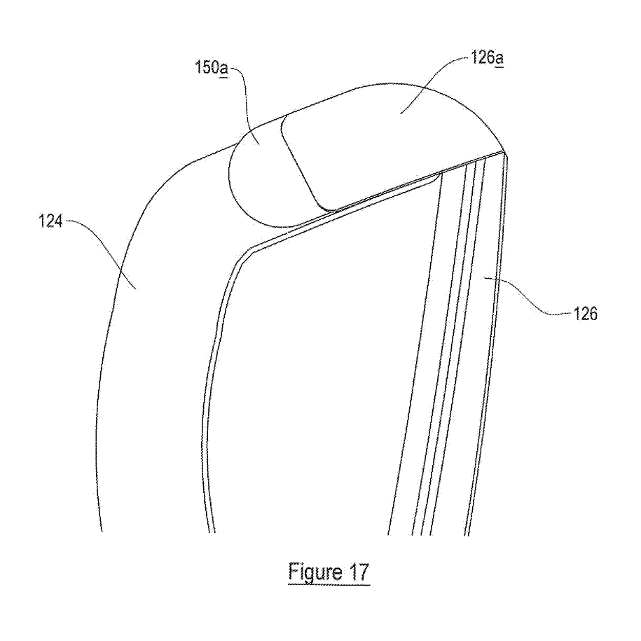

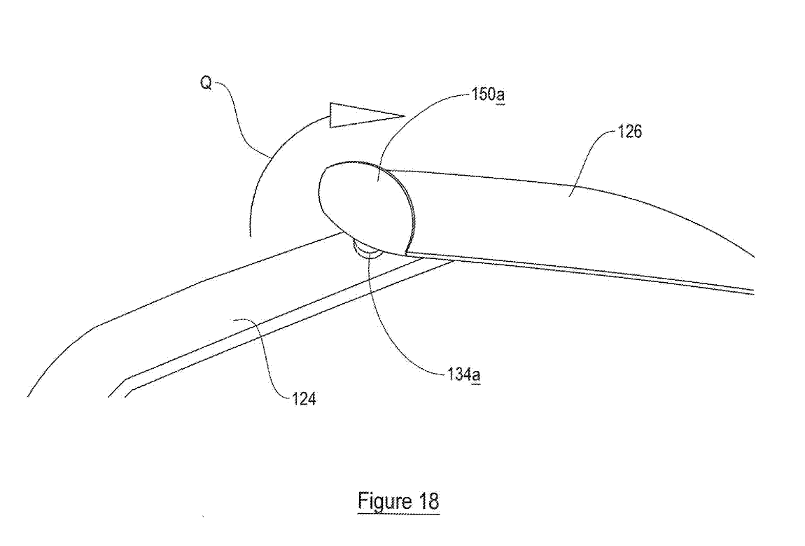

FIGS. 15 to 18 are close up end perspective views of component parts of the device of FIG. 11; and

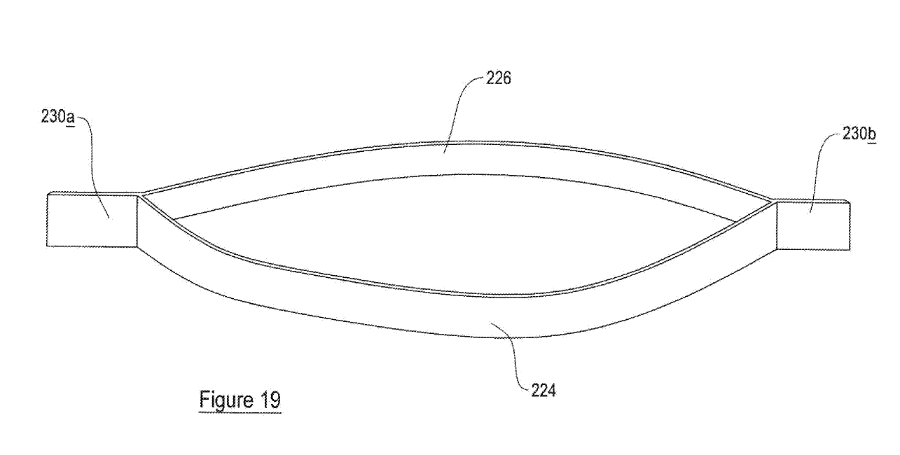

FIGS. 19 to 28 are various views of further embodiments of chamber opening support member for holding the opening of the chamber in its open condition.

Referring to the figures these show a wound management device 10 in accordance with the present invention for securing to the skin of a user around a wound site. The device 10 includes a generally oval securing element 12 which, is manufactured from a hydrocolloid material for adhesively securing the device 10 to a user. The element 12 is covered by a removable cover member which is removed prior to adhering the element 12. It is possible for the user or a health professional to cut or otherwise make an aperture 13 in the element 12 which follows closely a periphery of the wound. An example of such a cut can be seen in FIG. 3. It should be appreciated that whilst in the present example a hydrocolloid material has been used, other materials could also be utilised without departing from the scope of the invention.

The device 10 has a chamber defining member 14 which is connected to the element 12 for defining a chamber wherein the wound is accessible. In this particular embodiment the chamber defining member 14 is made from a generally see-through plastics material and is formed as a pair of wall elements 14a, 14b which are connected to each other at or near their peripheries, for example by adhesive or heat welding. The profile of the walls 14a, 14b can be seen in detail in FIGS. 2 and 3.

Advantageously, the device 10 in accordance with the present invention provides an opening 20 so that access can be gained to the chamber. In this particular embodiment the opening 20 is able to be closed when access is not necessary. In order to assist the user and/or a health professional in undertaking any procedures at the wound site e.g. cleaning of the wound, the present embodiment provides a pair of chamber opening support members 24, 26 which are configured for holding the opening 20 in its open condition. In the present example each member 24, 26 is a generally elongate plastic member which is connected (e.g. adhered or heat welded) to the element 14a such that the members 24, 26 are positioned opposite each other around the periphery of the opening 20. As shown in the figures, each member 24, 26 has regions 24a, 24b, 26a, 26b of reduced width (or thickness), which assist is permitting the members 24, 26 to flex with the user's body. Whilst in the present embodiment there are two of such regions on each member 24, 26, it should be appreciated that embodiments are envisaged having fewer or more such regions of reduced width or thickness.

In order to ensure that the opening 20 remains in its open condition, when desired, the present embodiment is provided with two connection members 30a, 30b. The function and configuration of the connection members 30a, 30b are identical, and thus only the connection member 30a will be discussed hereinafter.

The connection member 30a includes a part 31a which is pivotally connected at 32a to an outwardly facing side of the member 26. A free end of the part 31a is provided with a male connection formation 33 which is capable of connecting with a corresponding female connection formation 34a provided on an outwardly facing surface of the member 24. In this particular example the formations 33a, 34a are “pop” type fasteners although other appropriate means of fastening could be utilised. As can be seen from the figures, each connection member 30a, 30b connects a respective end of the member 26 to an adjacent respective end of the member 24. Referring particularly to FIGS. 1, 6 and 7, these show the device 10 with the connection members 30a, 30b in their connected positions. It can be seen that this provides for the maintenance of the opening 20 in its opening condition so that users can gain access therethrough.

The members 24, 26 are resiliently deformable (e.g. they can flex from their generally elongate positions to the generally curved position shown in FIGS. 1, 6 and 7) but they are also relatively stiff, which ensures that when the members 24, 26 are connected to each other by connection members 30a, 30b, they maintain the opening 20 in its open condition.

Referring particularly to FIGS. 7 and 8, it can be seen that the inwardly facing surface of each member 24, 26 is provided with a suitable sealing connection 25, 27 which can be connected to each other so as to close the opening and preferably seal the chamber from atmosphere. Seals of this kind are often known as zip locks.

It will also be seen on the figures that the device 10 includes a connector 40 which provides a fluid path into the chamber, for example for connecting a suction device or the like thereto. Such connections are well known in the art.

Whilst in the above embodiment the device 10 includes a pair of chamber opening support members 24, 26, it should be appreciated that embodiments are envisaged where there is only a single chamber opening support member which is capable of holding the opening in its open condition. For example, embodiments are envisaged including a single member which extends substantially completely around the periphery of the opening, with the member being divided into first and second portions which are connected to but movable relatively to one another (e.g. pivotally moveable). In such an embodiment only the free ends of the first and second portions need to be connected to one another to ensure that the opening remains in its open condition. In order to achieve this, a single connection member 30a or 30b could be provided to connect those free ends.

Referring to FIGS. 11 to 18, these show a second embodiment of a device 110 in accordance with the present invention. Features which correspond to those of the device 10 have been given the same reference numeral but with the addition of 100.

The device 110 is very similar to the device 10, with the exception of the means for holding the chamber opening support members 124, 126 in their open condition. In the device 110 the member 126 is longer than the member 124. At each of its ends it overhangs the members 124 by portions 126a, b. Connected to the end of each portion 126a, b is a user graspable extension portion 150a, b. A male connection formation 133a, b is provided on the position 126a, b which is capable of connecting with a corresponding female connection formation 134a, b provided on an outwardly facing surface of the member 124. In order to hold the device 110 in its open condition, the user must move the portion 126a, b in the direction of arrow P until the part 133a, b, engages the part 134a, b. To move the device 110 to its close condition, it is necessary to disconnect the members 124, 126 from each other, and this is achieved by the user pulling the extension part or tab 150a, b away from the member 124 in the direction of arrow Q. A user can simultaneous disconnect both ends of the member 126 from the member 124 by grasping one of the tabs 150a, b in each hand and pulling them away from each other.

Referring now to FIGS. 19 to 28, these show various views of alternative embodiments of chamber support opening member(s) for holding the opening of the chamber in its open condition. For ease of clarity and comparison, the remaining features of the device are not shown in these figures.

FIG. 19 shows an embodiment including a one-piece component part which forms a pair of members 224, 226. Ends of the members 224, 226 are connected to respective tab parts 230, 230b. Whilst not shown in FIG. 19, opposing surfaces of the members 224, 226 are provided with a means for connecting the members to each other so as to close the opening to the chamber. It should be noted that the component part shown is in its natural condition with the members 224, 226 held away from each other.

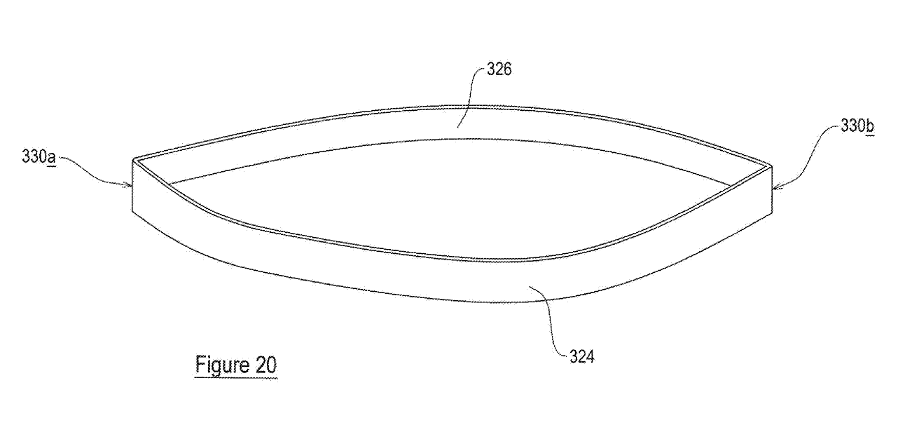

FIG. 20 is an embodiment similar to that shown in FIG. 19, but with the tab parts 230a, 230b omitted. Instead, the parts 324, 326 are connected at junctions 330a, 330b. Again the parts 324, 326 are held away from each other and the opposing inwardly facing surfaces of the parts 324, 326 are provided with suitable means to connect them to each other so as to seal the opening.

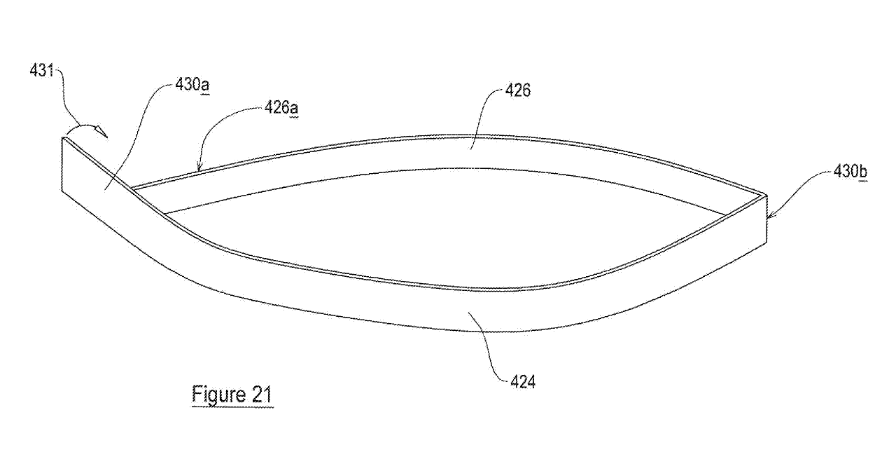

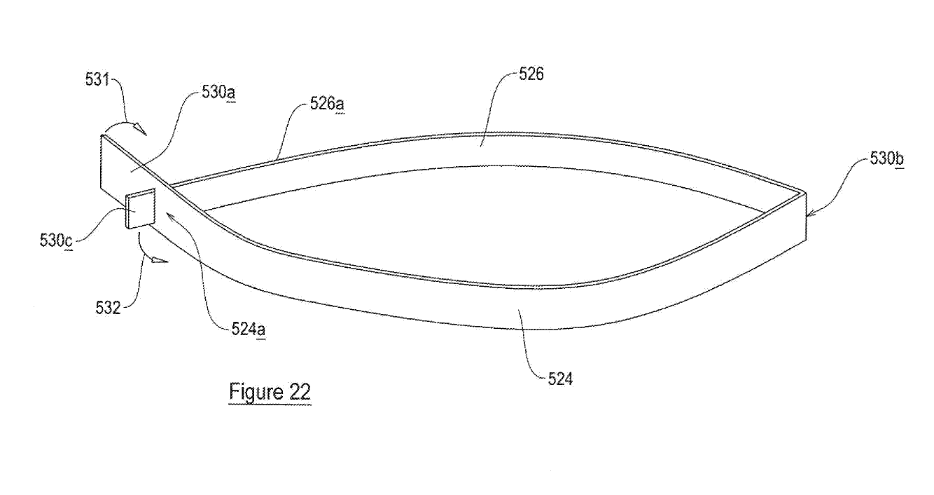

FIGS. 21 and 22 are further embodiments which are similar in construction to each other. The embodiment shown in FIG. 21 has members 424, 426 which are connected at respective ends by a junction 430b. A free end 430a of the member 424 is, in use, pivoted in the direction of arrow 431 so as to move the members 424, 426 away from each other. The free end 430a is then connected to the region 426a of the member 426 so as to hold the members 424, 426 away from each other.

In FIG. 22 a free end 530c of the member 526 extends through an aperture in the free end 530 of the member 524. Again, the free end 530a is pivoted in the direction of the arrow 531 and secured to a region 526a of the member 526 in order to hold the members 524, 526 away from each other in use.

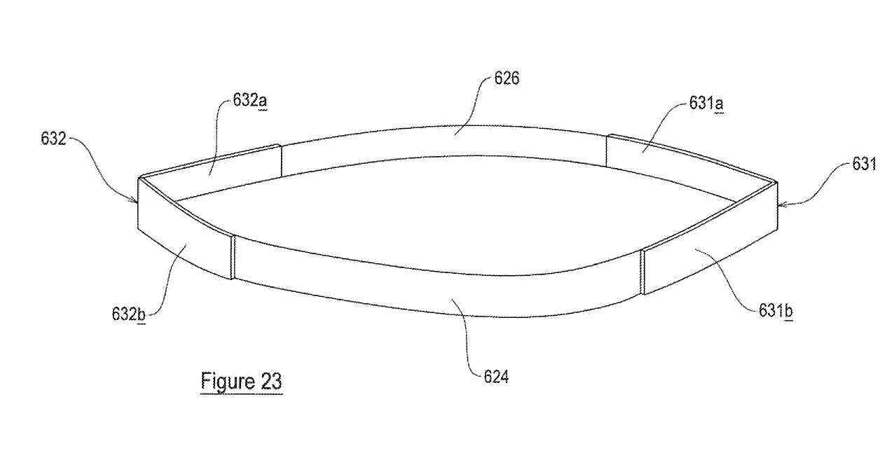

FIG. 23 shows a further alternative which is made from component parts 624, 626, 631, 632. The parts 631, 632 are mirror images of each other and are substantially v-shaped mouldings of a plastics material. Each part 631, 632 has limbs 631a, 631b, 632a, 632b. The member 624 extends between the limbs 631b, 632b, and the member 632 extends between the limbs 631a, 632a. The natural conditions of the parts are those shown in FIG. 23. In other words, the members 624, 626 are held away from each other in order to hold the opening of the chamber in an opening condition. As with the other embodiments suitable means is provided on the inwardly facing surfaces of the parts 624, 626 and the limbs 631a, 631b, 632a, 632b in order to close the opening.

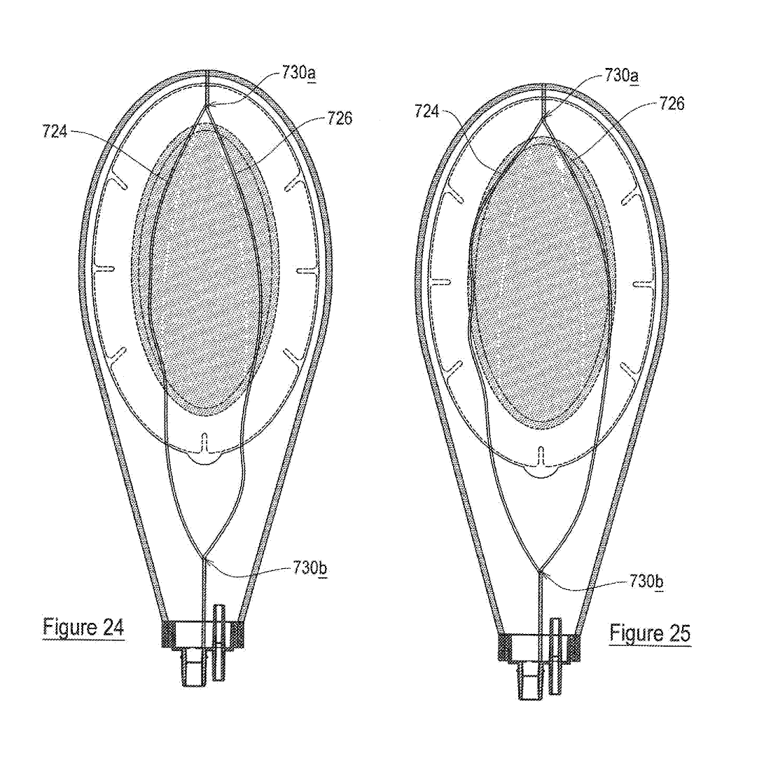

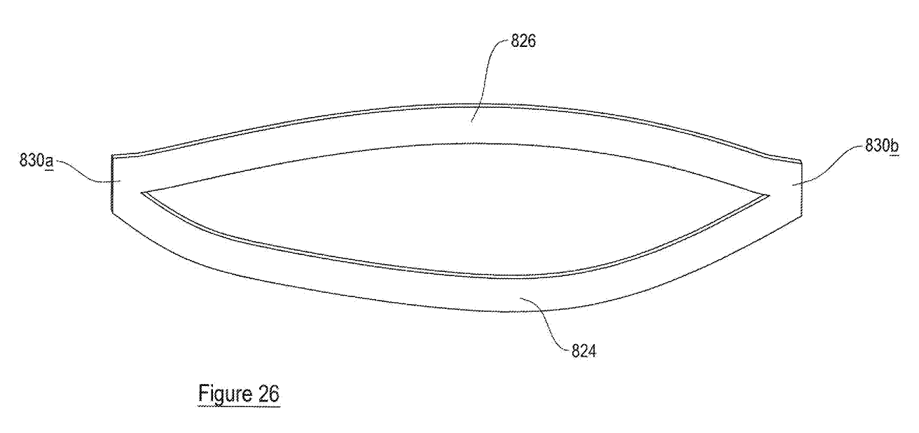

FIGS. 24 and 25 show an alternative embodiment where the members 724, 726 are made from a plastics material (e.g. a polyethylene homopolymer) which permits the members 724, 726 to be moulded to any desired condition. FIGS. 24 and 25 show alternative moulded conditions of the members 24, 26. This would permit a user to manoeuvre the members 724, 726 to any desired position in order to hold the opening to the chamber in an opening condition. As with the previous embodiments, a suitable means is provided on the inwardly facing surfaces of the members 724, 726 so that they can be secured to each other to close the opening. It should be appreciated that whilst in present embodiment the members 724, 726 are manufactured from a plastics material, it could be manufactured from any other suitable material, e.g. a material including or consisting completing of metal.

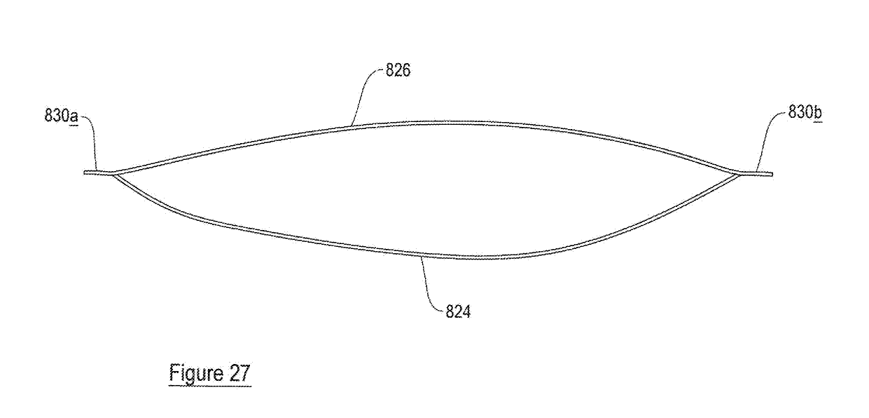

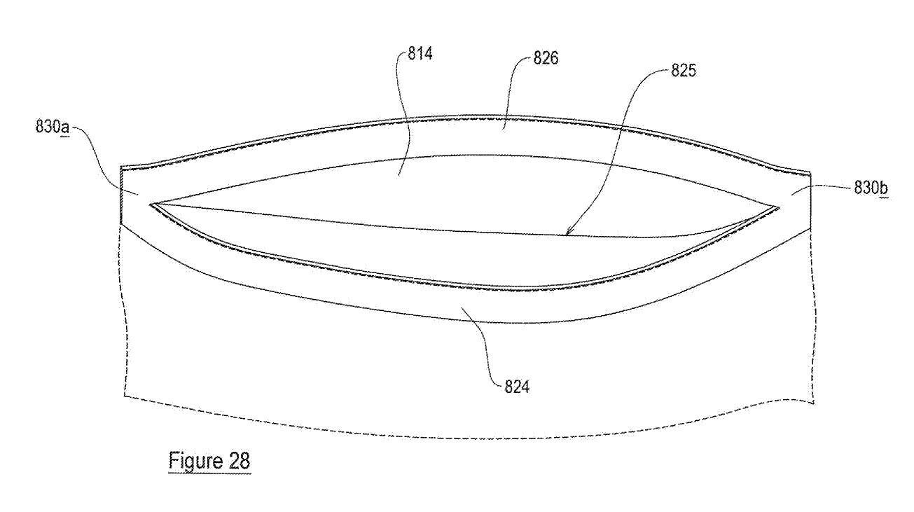

FIGS. 26 to 28 show a yet further embodiment. FIG. 26 is a perspective view, FIG. 27 is a planned view and FIG. 28 is a perspective view. In this embodiment the members 824, 826 are integrally moulded as a single component connected at ends 830a, 830b. The members 824, 826 are moulded such that in their natural condition (see FIG. 27), they are held away from each other so as to keep the opening of the chamber in its open condition. In order to close the opening the member 824 is moved towards the sheet of plastics material 814 where it connects to a suitable connection 825, which may for example be a zip lock or other suitable connection means so as to close the opening.

When used in this specification and claims, the terms “comprises” and “comprising” and variations thereof mean that the specified features, steps or integers are included. The terms are not to be interpreted to exclude the presence of other features, steps or components.

The features disclosed in the foregoing description, or the following claims, or the accompanying drawings, expressed in their specific forms or in terms of a means, for performing the disclosed function, or a method or process for attaining the disclosed result, as appropriate, may separately, or in any combination of such features, be utilised for realising the invention in diverse forms thereof.

1. A wound management device including:
at least one securing element for securing to skin around a wound site;
a chamber defining member connected to the at least one securing element for defining a chamber wherein the wound is accessible;
wherein an opening is provided in the chamber so that access can be gained to the chamber, said opening being moveable between closed and open conditions, and there being at least one member for holding the opening in its open condition.

2. A wound management device according to claim 1 including a chamber opening support member for holding the opening in its open condition.

3. A wound management device according to claim 2 including a connection member for connecting free ends of the chamber opening support member to each other, so as to hold the opening in its open condition.

4. A wound management device according to claim 3 wherein the chamber opening support member has first and second portions which are connected to each other, with the first portion having a free end and the second portion having a free end.

5. A wound management device according to claim 4 wherein the first and second portions are connectable to each other along their length so as to close, and preferably seal, the opening.

6. A wound management device according to claim 3 wherein, when the connection member is in its connected position, the chamber opening support member and the connection member extend around the complete periphery of the opening.

7. A wound management device according to claim 3 wherein the connection member is connected to one free end of the chamber opening support member and is releasably connectable to the other free end of the chamber opening support member.

8. A wound management device according to claim 3 wherein the connection member is pivotally connected to one free end of the chamber opening support member

9. A wound management device according to claim 1 including first and second chamber opening support members for holding the opening in its open condition.

10. A wound management device according to claim 9 wherein the first and second chamber opening support members are positioned around a periphery of the opening.

11. A wound management device according to claim 9 wherein the first and second chamber opening support members are positioned substantially opposite each other.

12. A wound management device according to claim 9 wherein the device includes a connection member for connecting a first end of the first chamber opening support member to a first end of the second chamber opening support member.

13. A wound management device according to claim 9 wherein the device includes two connection members, one for connecting a first end of the first chamber opening support member to a first end of the second chamber opening support member, and one for connecting a second end of the second chamber opening support member to a second end of the first chamber opening support member.

14. A wound management device according to claim 12 wherein, when the connection member is in its connected position, the first and second chamber opening support members and the connection members extend around the complete periphery of the opening.

15. A wound management device according to claim 9 wherein the first and second chamber opening support members are connectable to each other along their length so as to close, and preferably seal, the opening.

16. A wound management device according to claim 12 wherein the connection member is connected to a respective chamber opening support member and is releasably connectable to the other chamber opening support member.

17. A wound management device according to claim 12 wherein the connection member is pivotally connected to a respective chamber opening support member.

18. A wound management device according to claim 2 wherein the chamber opening support member is resiliently deformable.

19. A wound management device according to claim 1 wherein the chamber defining member includes at least two wall elements which are connected to each other.

20. A wound management device according to claim 19 wherein the at least two wall elements which are connected to each other at or near their peripheries to define the therebetween.

21. (canceled)

22. (canceled)
